(19) United States
(12) Patent Application Publication
(10) Pub. No.: US 2025/0230115 A1
(43) Pub. Date: Jul. 17, 2025

(54) REVAMP PROCESS FOR AN AMMONIA AND METHANOL CO-PRODUCTION PLANT

(71) Applicant: Topsoe A/S, Kgs. Lyngby (DK)

(72) Inventors: David Minguez, Copenhagen N (DK); Per Juul Dahl, Vedbæk (DK); Ameet Kakoti, Kokkedal (DK)

(73) Assignee: Topsoe A/S, Kgs. Lyngby (DK)

(21) Appl. No.: 18/853,992

(22) PCT Filed: Apr. 3, 2023
(86) PCT No.: PCT/EP2023/058604

§ 371 Date: Oct. 3, 2024

(30) Foreign Application Priority Data
Apr. 5, 2022 (DK) PA202200319

(51) Int. Cl. C07C 29/151; C07C 29/152

(52) U.S. Cl. CPC C07C 29/1518; C07C 29/152

(57) ABSTRACT

Process for revamp of a methanol and ammonia co-production plant.

The present invention relates to revamp of a process plant for the co-production of methanol and ammonia from a hydrocarbon feed. More particularly the invention is concerned with keeping the sequential and once-through (single pass) process for the production of methanol and ammonia product from a hydrocarbon containing feed stock by means of primary and secondary reforming, intermediary methanol and ammonia formation in a single process train, with a much-reduced production of excess of carbon dioxide and hydrogen.

From WO2011/020618 it is known to co-produce methanol, ammonia and urea from synthesis gas in a process employing an Air Separation Unit, with methanol production in the absence of nitrogen.

The invention concerns a revamp solution for a co-production plant capable of producing X tons of ammonia plus Y tons of methanol or Z tons of ammonia where Z is equal to X+Y within plus/minus 10%.

The inventive revamp solution results in a process capable of producing Z tons of ammonia plus y tons of methanol

The revamp invention ensures minimum capex as it can be made utilizing all the existing equipment by:
adding a parallel gas cleaning reactor or accept shorter lifetime of the catalyst in the existing reactors
adding additional reforming capacity by adding a heat exchange reformer (HTER™) alternatively an SMR or a SMR-B or a convection reformer (HTCR™)
adding a once through methanol synthesis in parallel to the existing shift and CO2 removal steps.

The invention ensures that the main part of the plant (>90% of capex) remain unchanged for a capacity increase from X+Y to Z+Y where Z=X+Y+/−10%.

It is the general object of the invention to maintain a process for co-producing methanol and ammonia with much reduced production of excess of carbon dioxide and hydrogen from a hydrocarbon feed stock after being revamped.

The term “much reduced production of excess of carbon dioxide and hydrogen” shall be understood in such a manner that conversion of the hydrocarbon feed stock to synthesis gas is performed at conditions to utilise part of the CO2 related to ammonia production from hydrocarbon for methanol production and to utilise excess hydrogen production in connection with methanol production for ammonia production, resulting in less emission of carbon dioxide and hydrogen only as required for purging of inert gases from the co-production of methanol and ammonia.

The general object of the invention is achieved by installing a HTER™ to increase the syngas production in the reforming step and by installing a once through methanol synthesis in parallel to the existing shift and CO₂ removal section.

Accordingly, the invention provides a process for co-producing methanol and ammonia from a hydrocarbon feedstock comprising the sequential steps of:
(a) producing a synthesis gas from hydrocarbon feedstock containing hydrogen, carbon monoxide and carbon dioxide and nitrogen by steam reforming the hydrocarbon feedstock in a primary reforming stage and subsequently in a secondary reforming stage;
(b) subjecting the synthesis gas from step (a) to a partial water gas shift;
(c) removing at least part of the carbon dioxide from the synthesis gas from step (b);
(d) catalytically converting the carbon monoxide, carbon dioxide and hydrogen of the synthesis gas from step (c) in a once-through methanol synthesis stage and withdrawing an effluent containing methanol and a gaseous effluent comprising nitrogen, hydrogen and unconverted carbon monoxide and carbon dioxide;
(e) subjecting the gaseous effluent from step (d) to catalytic methanation to remove the unconverted carbon monoxide and carbon dioxide;
(f) catalytically converting the nitrogen and hydrogen in the gaseous effluent from step (e) in an ammonia synthesis stage and withdrawing an effluent containing ammonia and an off-gas stream comprising hydrogen, nitrogen and methane;
wherein part of the synthesis gas from step (a) is send through a once-through methanol synthesis stage (g) where an effluent containing methanol is withdrawn and where the gaseous effluent containing nitrogen, hydrogen and unconverted carbon monoxide and carbon dioxide is send to step (d);
and wherein the synthesis gas capacity of step (a) is increased by adding a heat exchange reformer (HTER) using the sensitive heat in the secondary reformer outlet gas to reform additional hydrocarbon feed and thereby produce more synthesis gas

The increased synthesis gas capacity of step (a) can alternatively be obtained by installing a parallel reforming stage, such as SMR or a SMR-B or a HTCR™

As used herein the term “partial water gas shift of the synthesis gas” means that a part of synthesis gas is by-passed the water gas shift reaction and combined with the shifted synthesis gas after the reaction.

As further used herein the term “primary reforming stage” means reforming being conducted in a conventional steam methane reformer (SMR), i.e. tubular reformer with the heat required for the endothermic reforming being provided by radiation heat from burners, such as burners arranged along the walls of the tubular reformer.

As also used herein the term “secondary reforming stage” means reforming being conducted in an autothermal reformer or catalytic partial oxidation reactor, both using air as combustion medium.

As also used herein the term HTER™ means a heat exchange reformer which gets the required energy by cooling the process gas outlet the existing reforming step

As also used herein the term SMR-B means a steam methane reformer using bayonet tubes.

As also used herein the term HTCR™ means a heat exchange reformer which gets the required energy by cooling hot flue gas originating from combustion of hydrocarbon fuel

As further used herein, the term “once-through methanol synthesis stage” means that methanol is produced in at least one catalytic reactor operating in a single pass configuration, i.e. without significant recirculation (not more than 5%) of the volume flow of any gas produced in the methanol synthesis back to the at least one methanol reactor of the methanol synthesis stage, particularly the gas effluent containing hydrogen and unconverted carbon oxides.

Suitable hydrocarbon feed stocks for use in the invention include methane, natural gas, naphtha and higher hydrocarbons.

Preferably the hydrocarbon feedstock comprises methane, for instance in the form of natural gas, liquefied natural gas (LNG) or substitute natural gas (SNG).

When employing naphtha and higher hydrocarbons, it is preferred to subject these feed stocks to a prereforming step prior to the primary reforming stage. However, prereforming can be employed for all types of hydrocarbon feed stock.

By the invention we make direct use of the reactions governing reforming, methanol synthesis and ammonia synthesis so that methanol and ammonia can be co-produced without the necessity to remove excess nitrogen from the synthesis gas. This is ensured by adjusting the split between steam reforming and secondary reforming

The control of the carbon monoxide/carbon dioxide ratio to meet the required amount of nitrogen, carbon monoxide, carbon dioxide and hydrogen for the methanol and ammonia synthesis is obtained by subjecting part of the synthesis gas to the water gas shift reaction prior to the removal of carbon dioxide in step (c) and by controlling the part of synthesis gas send through the new methanol synthesis.

The final synthesis gas is by the above measures adjusted to contain carbon monoxide, carbon dioxide, hydrogen and nitrogen in a molar ratio substantially complying to the stoichiometric amounts in the ammonia synthesis

Removal of carbon dioxide from the secondary reformed synthesis gas may be performed by any conventional means in a physical or chemical wash as known in the art.

The methanol synthesis stages are preferably conducted by conventional means by passing the synthesis gas at high pressure and temperatures, such as 60-150 bar and 150-300° C. through at least one methanol reactor containing at least one fixed bed of methanol catalyst.

A particularly preferred methanol reactor is a fixed bed reactor cooled by a suitable cooling agent such as boiling water, e.g. boiling water reactor (BWR).

In a specific embodiment the methanol synthesis stage in step (d) is conducted by passing the synthesis gas through a series of one or more boiling water reactors and subsequently through an adiabatic fixed bed reactor.

Accordingly, the invention enables the operation of the methanol and ammonia synthesis section at similar operating pressures, for instance 130 bar, which implies a simplified process with significant savings in size of equipment as mentioned above. Yet it is also possible to operate at two different operating pressures, for instance 80-90 bar in the methanol synthesis stage and 130 bar in the ammonia synthesis stage, which implies energy savings in the methanol synthesis stages.

The effluent streams containing methanol are preferably liquid effluents. These effluents are obtained by cooling and condensation of the synthesis gas from the methanol reactors. Accordingly the process of the invention may further comprise cooling the synthesis gas withdrawn from each methanol reactor to condense methanol and passing the gas through a separator, withdrawing a bottom fraction from the separator containing the raw methanol, withdrawing an overhead fraction containing synthesis gas which is passed to the subsequent methanol reactor or step (d) or (e), and forming a single liquid effluent containing methanol by combining the bottom fractions of the separators of each reactor containing the raw methanol.

It would be understood that the term “methanol reactor” as used herein encompasses adiabatic fixed bed reactors and cooled reactors such as boiling water reactors and reactors of the condensing-methanol type which comprises within a common shell a fixed bed of methanol catalyst particles and cooling means adapted to indirectly cooling the methanol synthesis gas with a cooling agent.

In step (e) the catalytic methanation stage for conversion of carbon monoxide to methane is conducted in at least one methanation reactor, which is preferably an adiabatic reactor containing a fixed bed of methanation catalyst.

In step (f) the ammonia synthesis gas from the methanation stage containing the correct proportion of hydrogen and nitrogen (H₂: N₂ molar ratio between 2.9:1 and 3.1:1) is optionally passed through a compressor to obtain the required ammonia synthesis pressure, such as 120 to 200 bar, preferably about 130 bar. Ammonia is then produced in a conventional manner by means of an ammonia synthesis loop comprising at least one ammonia converter containing at least one fixed bed of ammonia catalyst, with interbed cooling. Ammonia may be recovered from the effluent containing ammonia as liquid ammonia by condensation and subsequent separation. Preferably, an off-gas stream containing hydrogen, nitrogen and methane is withdrawn from the ammonia synthesis stage, as also is a hydrogen-rich stream (>90 vol % H₂). These streams may for instance stem from a purge gas recovery unit. Preferably, this hydrogen stream is added to the methanol synthesis stage (step (c)), for instance by combining with the methanol synthesis gas. The recycle of this hydrogen-rich stream enables a higher efficiency in the process as useful hydrogen is utilised in the methanol synthesis and subsequent ammonia synthesis rather than simply being used as fuel.

In order to improve the energy efficiency of the process the off-gas stream containing hydrogen, nitrogen and methane of step (e) is returned to step (a), i.e. it is returned as off-gas fuel to the reforming section of the plant, specifically to the primary reforming stage.

The ammonia being withdrawn from the ammonia synthesis can partly be converted to urea product by reaction with carbon dioxide recovered from step (c) as described above.

1. Process for revamp of a co-producing methanol and ammonia plant comprising the sequential steps of:
(a) producing a synthesis gas from hydrocarbon feedstock containing hydrogen, carbon monoxide and carbon dioxide and nitrogen by steam reforming the hydrocarbon feedstock in a primary reforming stage and subsequently in a secondary reforming stage;
(b) subjecting the synthesis gas from step (a) to a partial water gas shift;
(c) removing at least part of the carbon dioxide from the synthesis gas from step (b);
(d) catalytically converting the carbon monoxide, carbon dioxide and hydrogen of the synthesis gas from step (c) in a once-through methanol synthesis stage and withdrawing an effluent containing methanol and a gaseous effluent comprising nitrogen, hydrogen and unconverted carbon monoxide and carbon dioxide;
(e) subjecting the gaseous effluent from step (d) to catalytic methanation to remove the unconverted carbon monoxide and carbon dioxide;
(f) catalytically converting the nitrogen and hydrogen in the gaseous effluent from step (e) in an ammonia synthesis stage and withdrawing an effluent containing ammonia and an off-gas stream comprising hydrogen, nitrogen and methane;
wherein part of the synthesis gas from step (a) is send through a once-through methanol synthesis stage (g) where an effluent containing methanol is withdrawn and where the gaseous effluent comprising nitrogen, hydrogen and unconverted carbon monoxide and carbon dioxide is send to step (d);
and wherein the synthesis gas capacity of step (a) is increased by adding a heat exchange reformer using the sensitive heat in the secondary reformer outlet gas to reform additional hydrocarbon feed and thereby produce more synthesis gas.

2. Process according to claim 1 wherein the hydrocarbon feedstock is natural gas, substitute natural gas, naphtha and higher hydrocarbons.

3. Process according to claim 1, wherein the methanol synthesis stage in step (d) is conducted by passing the synthesis gas through a series of one or more boiling water reactors and subsequently through an adiabatic fixed bed reactor.

4. Process according to claim 3, wherein the one or more boiling water reactor is in the form of a single reactor of the condensing-methanol type which comprises within a common shell a fixed bed of methanol catalyst particles and cooling means adapted to indirectly cooling the methanol synthesis gas with a cooling agent.

5. Process according to claim 3, further comprising cooling the synthesis gas withdrawn from each methanol reactor to condense methanol and passing the remaining synthesis gas through a separator, withdrawing a bottom fraction from the separator containing the raw methanol, withdrawing an overhead fraction containing synthesis gas which is passed to the subsequent methanol, and forming a single liquid effluent containing methanol by combining the bottom fractions of the separators of each reactor containing the raw methanol.

6. Process according to claim 1, wherein the methanol synthesis stage in step (g) is conducted by passing the synthesis gas through a series of one or more boiling water reactors.

7. Process according to claim 6, wherein the one or more boiling water reactor is in the form of a single reactor of the condensing-methanol type which comprises within a common shell a fixed bed of methanol catalyst particles and cooling means adapted to indirectly cooling the methanol synthesis gas with a cooling agent.

8. Process according to claim 6, further comprising cooling the synthesis gas withdrawn from each methanol reactor to condense methanol and passing the gas through a separator, withdrawing a bottom fraction from the separator containing the raw methanol, withdrawing an overhead fraction containing synthesis gas which is passed to the subsequent methanol, and forming a single liquid effluent containing methanol by combining the bottom fractions of the separators of each reactor containing the raw methanol

9. Process according to claim 1, wherein the off-gas stream in step (e) containing hydrogen, nitrogen and methane is employed as fuel for heating the reforming stage in step (a).

10. Process according to claim 1, wherein the increased synthesis gas capacity of step (a) is obtained by installing a parallel reforming stage.

11. Process according to claim 10, wherein the parallel reforming step is a tubular reformer or a baronet type reformer or a heat exchange reformer.

12. Process according to claim 1, wherein the hydrocarbon feed stock is subjected to pre-reforming upstream of step (a).

13. Process according to claim 1, wherein the hydrocarbon feedstock is purified for contaminants comprising sulphur containing components upstream step (a) and send through an optional pre-reforming step prior to step (a).
